(19) United States
(12) Patent Application Publication
(10) Pub. No.: US 2019/0336702 A1
(43) Pub. Date: Nov. 7, 2019

(54) SYRINGE NEEDLE RETAINER AND CAP ASSEMBLY

(71) Applicant: Kaveh Karimi, Clovis, CA (US)

(72) Inventor: Kaveh Karimi, Clovis, CA (US)

(21) Appl. No.: 16/378,856

(22) Filed: Apr. 9, 2019

Related U.S. Application Data
(60) Provisional application No. 62/665,245, filed on May 1, 2018.

(51) Int. Cl. A61M 5/32; A61M 5/24

(52) U.S. Cl. CPC A61M 5/3269; A61M 5/3204; A61M 2005/2481; A61M 2005/2407; A61M 5/2459

(57) ABSTRACT

A syringe cap and needle assembly includes a needle retainer, a needle member secured within the needle retainer, and a cap. The needle member further includes an exterior needle end that extends outside of a forward end of the needle retainer. The cap is configured to slideably move with respect to the needle retainer between at least a first position and a second position. in the first position, the cap substantially surrounds the exterior needle end. In the first position and the second position, the cap substantially surrounds at least a portion of the needle retainer. in the second position, at least a portion of the exterior needle end is not surrounded by the cap.

### CROSS REFERENCE TO RELATED APPLICATION

This application claims priority to and the benefit of U.S. Provisional Appln. No. 62/665,245, filed on May 1, 2018, the disclosure of which is incorporated herein by reference in its entirety.

### BACKGROUND

The present invention is directed generally to syringes for hypodermic needles, and in particular an improved syringe body, syringe needle assembly, and cap assembly that covers a syringe needle assembly with a cap before, during, and/or after use to prevent injury from the needle and possible exposure of the needle to contamination or disease before, during, and/or after use.

The present improved syringe body, syringe needle assembly, and cap assembly is disclosed herein in the context of syringes which are configured to enable the use of prepared ampoules, which ampoules contain a measured amount of medicinal or other liquid, and which are inserted into the syringe, and removed and discarded after use, and in particular to an improved needle assembly and safety cap assembly. The present improved syringe body, syringe needle assembly, and cap assembly is also disclosed herein in the context of syringes which are configured for separating and ejecting a needle assembly from a syringe body without the operator having to directly handle a potentially contaminated used needle assembly, in particular by transferring a force from a plunger of the syringe to the needle assembly when the syringe does not have an ampoule therein. However, the present invention is not considered so and could be applied to syringes of other types, including but not limited to those which use removable prepared ampoules and/or those which have an easily removable needle assembly.

A typical prior art syringe, which employs exchangeable ampoules may be used by an operator to perform injections. In some instances, an ampoule may not have enough medication stored therein to sufficiently medicate a patient as desired. Accordingly, syringes employing exchangeable ampoules may be used. If a first exchangeable ampoule is emptied of medication before desired injections for a given patient are completed, the first ampoule is replaced by a second ampoule without changing out the needle, so that the injection(s) may continue without significant interruption. However, the prior art syringes that provide for exchangeable ampoules (formed as a glass cylinder having a puncturable seal at one end, comprises a syringe body, which may be a hollow substantially cylindrical shell, which has openings at its forward and rearward ends, as well as at least one lateral opening) lack a secure way to protect the operator from the exposed needle while the ampoules are exchanged, and the prior art syringes further lack a way to protect the needle from contamination while the ampoules are exchanged. A plunger assembly is attachable to the rearward end of the syringe body. The plunger assembly usually includes an outer collar which may be internally threaded, so as to screw onto external threads on the rearward end of the syringe body. A plunger shaft is centered within the collar, and capable of sliding axially relative to the collar. On the forward end of the plunger shaft a hook, which emanates from a disk having a diameter slightly greater than that of the plunger shaft, is provided to engage the plunger piston in the ampoule. At the rearward end of the plunger shaft, a thumb ring or other gripping device is provided to enable both forward and rearward force to be applied by the operator.

A typical prior art syringe, which employs an easily removable needle assembly may also be used by an operator to perform injections. For example, certain syringes have reusable bodies while the needle and needle assembly in which the needle is secured is removed and disposed of after use. However, the prior art syringes that have removable needle assemblies lack a secure way to protect the operator from the exposed needle while the needle assembly is actually being ejected from the syringe body and disposed of.

Accordingly, it is desirable to provide a syringe body, syringe needle assembly, and cap assembly which is adapted to protect an operator from a needle while ampoules are interchanged and while a needle assembly is ejected and disposed of. In addition, it is desirable to provide a syringe body, syringe needle assembly, and cap assembly which is adapted to protect a needle from contamination while ampoules are interchanged.

It is further desired to provide a syringe body, syringe needle assembly, and cap assembly in which a needle assembly is configured to be mated to a syringe body easily. Conventional cap apparatuses are applied to the needle assembly by approaching the assembly directly toward the exposed needle point, which presents an undesirable risk of injury.

Accordingly, an additional consideration is that the needle tip should be sheathed prior to mounting upon the syringe body, to prevent accidental injury to the operator or patient, and also to maintain the needle in a sterile condition as long as possible, but also after use, again to prevent injury, and to also preclude possible transmission of disease from the contaminated needle assembly. An additional consideration is that the needle tip should be sheathed at all times while ampoules are being interchanged. An additional consideration is that the operator should not have to move their hand toward and risk contacting an exposed needle point in order to sheath the needle either before use, during interchanging of ampoules, or after use of the needle.

It is thus an object of the invention to provide for an improved needle cap assembly construction which provides for safely and securely sheathing a needle before use, during interchanging of ampoules, and after use of the needle.

Yet another object of the invention is to provide for an improved needle cap assembly construction which provides for protection of a needle from contamination while ampoules are interchanged.

These and other objects of the invention will become apparent in light of the present specification, claims, and drawings.

### SUMMARY OF THE INVENTION

The present invention is an improved syringe body, syringe needle assembly, and cap assembly that is disclosed herein in the context of a typical syringe apparatus of the type which utilizes interchangeable ampoules.

A syringe body apparatus comprises a forward end which is configured for receiving a syringe needle assembly. When the syringe needle assembly is operably affixed to the syringe body, a plunger of the syringe may be actuated to drive medication out of an ampoule through a needle of the needle assembly. The syringe needle assembly comprises a needle and a needle retainer that secures the needle. The needle retainer engages with the syringe body to affix the needle assembly to the syringe body.

A cap assembly comprises an inner cap and an outer cap. The inner cap is positioned around an unused needle and attached to a needle assembly before use. The outer cap is positioned around both an unused needle and attached to a needle assembly before use. The outer cap includes a guide pin on its interior surface that rests in a locking channel on an outside surface of the needle retainer before the needle assembly is affixed to a syringe body. In other words, the outer cap extends around the inner cap, the needle, and a portion of the needle retainer, with a guide pin of the outer cap resting in a locking channel of the needle retainer. The guide pin and locking channel may interact to keep the outer cap affixed to the needle retainer.

In order to affix the needle assembly, inner cap, and outer cap to a syringe body, an operator may grasp the outer cap and press the needle assembly onto the syringe body. That is, pressing on the outer cap transfers force into the needle assembly via the interaction of the guide pin of the outer cap and the locking channel of the needle retainer, which presses the needle assembly onto the syringe body.

The outer cap, after the needle assembly is affixed to the syringe body, is rotated such that the guide pin of the outer cap moves through the locking channel of the needle retainer into a release channel of the needle retainer. Once the guide pin is in the release channel, the outer cap is movable toward the syringe body (e.g., toward a rear end of the syringe), and the guide pin passes into a release channel located on an exterior surface of the syringe body and eventually into a locking channel of the syringe body. The guide pin is moved into a locking channel of the syringe body so that the outer cap will stay in place while the needle is used for injecting medication and while the inner cap is removed. The outer cap is further configured with an opening at its forward end, such that as the guide pin moves into the release channel of the syringe body, the inner cap surrounding the needle is exposed.

The inner cap is affixed to the needle retainer, for example, through an interference fit. The inner cap may be removed, exposing the needle, after the outer cap is moved to expose the inner cap. Once the inner cap is removed, the syringe may be used to inject medication in a patient as desired by the operator. In the event an ampoule of the syringe runs out of medication before the operator desires to continue delivering medication, the outer cap may be moved back to a position that covers the needle while the ampoules are interchanged. In other words, the guide pin of the outer cap may be moved back through the release channels of the syringe body and the needle retainer so that the outer cap moves toward the forward end of the syringe, thereby covering the needle again. The guide pin may also be moved back into the locking channel of the needle retainer so that the outer cap does not move while the ampoules are interchanged. Once the ampoules are successfully interchanged, the outer cap may be moved back through the release channels of the syringe body and the needle retainer so that the outer cap exposes the needle again for injections performed by the operator.

When injections are complete (whether an operator exchanged ampoules or not), the outer cap may be moved to cover the needle again. In doing so, the guide pin is moved through the release channels of the syringe body and the needle retainer and into the locking channel of the needle retainer. The needle retainer may also have permanent locking means to secure the guide pin permanently once the operator is ready to dispose of a needle. For example, a locking channel of the needle retainer may include a retaining catch that allows the guide pin to move past the retaining catch, but once the guide pin does move past the retaining catch it cannot move past the retaining catch again. In this way, the outer cap may be locked in position to the needle retainer such that the needle is covered and the needle assembly may be ejected from the syringe body safely. In addition, as the needle assembly is ejected from the syringe body, the outer cap is also ejected and stays securely covering the needle because the outer cap is permanently affixed to the needle retainer.

### BRIEF DESCRIPTION OF THE DRAWINGS

FIG. 1 is a partially exploded elevational view, partly in section, of a prior art syringe apparatus;

FIG. 2 is a side elevational view, partially in section, of an assembled syringe apparatus according to one embodiment of the present invention;

FIG. 3 is an exploded perspective view of the syringe apparatus according to the embodiment of the invention of FIG. 2;

FIG. 4A is a perspective view of a needle and cap assembly according to one embodiment of the invention;

FIG. 4B is a side view of a guide pin on a side wall of an outer needle cap according to one embodiment of the invention;

FIG. 5 is a perspective view of a needle assembly according to the embodiment of the invention of FIG. 4A;

FIG. 6 is a perspective view, partly in section, of a portion of a syringe body according to the embodiment of the invention illustrated in FIG. 3;

FIG. 7 is a sectional view of a portion of the syringe apparatus according the embodiment of the invention of FIG. 2; and

FIG. 8 is an exploded perspective view of a syringe body, a needle assembly, and an outer needle cap according to one embodiment of the present invention.

### DETAILED DESCRIPTION

While this invention is susceptible of embodiment in many different forms, there is shown in the drawings and will be shown in detail herein, several specific embodiments, with the understanding that the present disclosure is to be considered as an exemplification of the principles of the invention and is not intended to limit the invention to the embodiments illustrated.

### The Prior Art

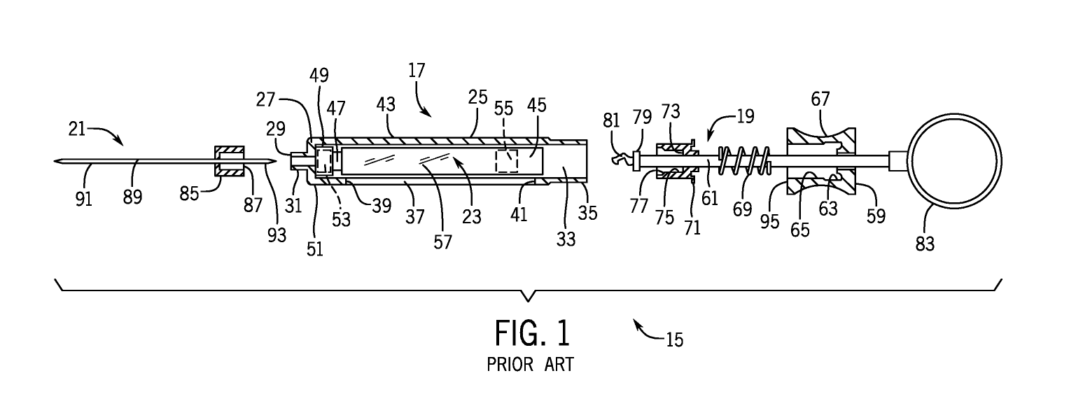

A prior art syringe 15 is shown in partially exploded sectional view in FIG. 1. Syringe 15 is composed of syringe body 17, plunger assembly 19, needle assembly 21, and interchangeable, disposable ampoule 23.

Syringe body 15 is formed as a substantially hollow cylinder 25, having a substantially closed forward end 27 with coupling 29 having thread 31, and a substantially open rear end 33 with thread 35. In addition, a lateral aperture 37, bounded by front edge 39 and rear edge 41, is provided. Lateral aperture 37 has a width (not illustrated) which is sufficient to accommodate the width of ampoule 23, but has a length which is less than that of ampoule 23.

Ampoule 23, which has a conventional configuration, is composed of cylinder 43, which may be fabricated from glass, plastic or other substantially transparent, sterilizable material. Cylinder 43 has a substantially open rear end 45, and a necked front end 47, around which a collar 49, which may be of metal, is affixed. The forward face of collar 49 has a central circular aperture 51. A puncturable seal 53, which may be formed from rubber, plastic or the like, is arranged underneath collar 49, and between collar 49 and necked front end 47, and centered relative to and spanning across the central circular aperture 51 in collar 49. Plunger piston 55 is arranged to fit, in a slightly forced manner, within cylinder 43 and seal off rear end 45 of cylinder 43. Plunger piston 55 is preferably fabricated from a sterilizable rubber material, or the like, which is capable of being pushed or pulled along the interior of cylinder 43 without losing its sealing integrity. To facilitate movement of plunger piston 55, a lubricant material may be disposed in circumferential grooves (not shown) around plunger piston 55, between plunger piston 55 and cylinder 43. Medicinal liquid 57 is sealed in ampoule 23 between seal 53 and plunger piston 55. The diameters of cylinder 25 and of cylinder 43 are such that ampoule 23 is capable of sliding or being propelled freely, once ampoule 23 is within cylinder 25.

Plunger assembly 19 is composed of outer collar 59, through which plunger shaft 61 is configured to freely slide. Outer collar 59 includes central well 63, on the inner surface of which are formed thread 65. Curved grip contour 67 is provided to facilitate grasping of syringe 15, for example, by the index and middle fingers of the operator, once syringe 15 has been assembled. Also surrounding plunger shaft 61 is helical spring 69 and inner collar 71. Inner collar 71 is configured to fit closely around plunger shaft 61 at inner flange 73. Inner collar 71 is also provided with a central well 75, and an inner ledge 77. The outer diameter of inner collar 71 is less than the inner diameter of central well 63 of outer collar 59. Disk 79 has a diameter greater than the inner diameter of inner flange 73, but less than the inner diameter of central well 75. Hook 81 is configured to be sufficiently sharp to be able to pierce into and engage plunger piston 55. To facilitate manipulation of plunger shaft 61, thumb ring 83 is provided, so that plunger shaft 61 may be pushed or pulled with only one-handed use by the operator.

Needle assembly 21 comprises a cap-like needle retainer 85, which has thread 87 formed therein which mate with thread 31 of coupling 29. Hollow needle 89 passes through needle retainer 85 and includes exterior end 91 and interior end 93. Both exterior and interior ends 91 and 93, respectively, have sharp beveled points, for facilitating entry into the patient, and for piercing seal 53, respectively.

To assemble syringe 15, outer collar 59 is screwed onto thread 35 of syringe body 17. When fully attached, the forward flange 95 of curved grip contour 67 is positioned substantially immediately adjacent rear edge 41 of aperture 37. Needle assembly 21 is then screwed onto coupling 29. In its assembled unloaded resting position, for example, when syringe 15 is held with the exterior end 91 of needle 89 pointing upward, inner flange 73 of inner collar 71 is approximately level with rear edge 41, and pusher disk 79 is at the bottom of central well 75, so that hook 81 is completely “below” rear edge 41. While the rear end 45 of ampoule 23 may be angled through aperture 37, and into contact with inner ledge 77 of inner collar 71, front edge 39 of aperture 37 prevents ampoule 23 from being fully inserted into syringe body 17. By pulling backward with thumb ring 83 on plunger shaft 61, against the force of spring 69, however, pusher disk 79 forces inner collar 71 to recede, permitting ampoule 23 to follow, and thus clear front edge 39 of aperture 37. When thumb ring 83 is released, spring 69 then forces inner collar 71 against rear end 45 of ampoule 23 forward toward forward end 27 of syringe body 17, and tending to force interior end 93 of needle 89 against and through seal 53. The piercing process is completed when thumb ring 83 is pressed forward, with hook 81 piercing into, but preferably not through, plunger piston 55, and with pusher disk 79 pushing against plunger piston 55. An additional slight push on thumb ring 83 will then cause some of the liquid to be driven into needle 89, and out exterior end 91, in the customary manner for preparing a syringe for positioning and injection.

The procedure for removal and exchange of the ampoule 23, after use, and the attendant potential problems, having already been described, they will be only briefly summarized here. Essentially, in the process of withdrawing plunger shaft 61, to bring it out from “within” ampoule 23, ampoule 23 will remain in its forward position within syringe body 25, even when plunger shaft 61 has been drawn back sufficiently to cause disk 79 to pull inner collar 71 away from ampoule 23. This is due to the gripping force seal 53 exerts upon interior needle end 93. The result is the necessity of the operator to use both hands in order to free the ampoule, which is undesirable with respect to efficiency and hygienic safety. An additional drawback of prior art syringe 15 is the need to use two hands in the removal of the used needle assembly which is also to be discarded, for the same reasons of hygienic safety and efficiency.

### The Improved Needle Retainer and Cap Assembly

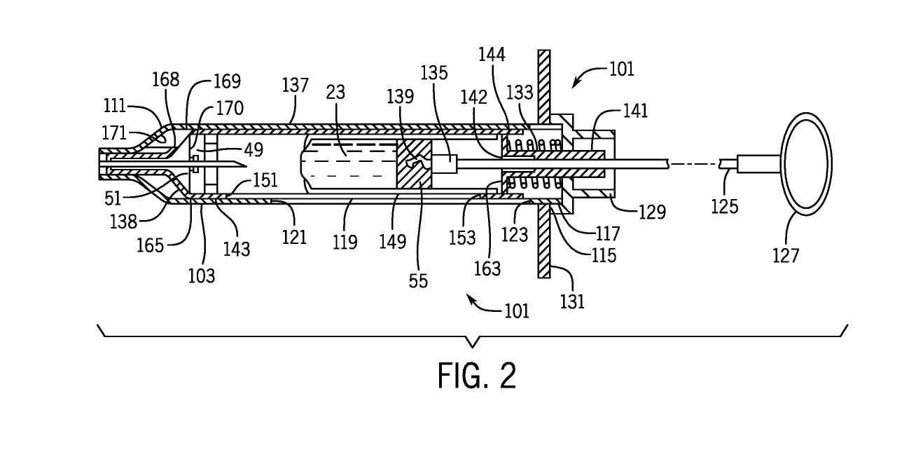

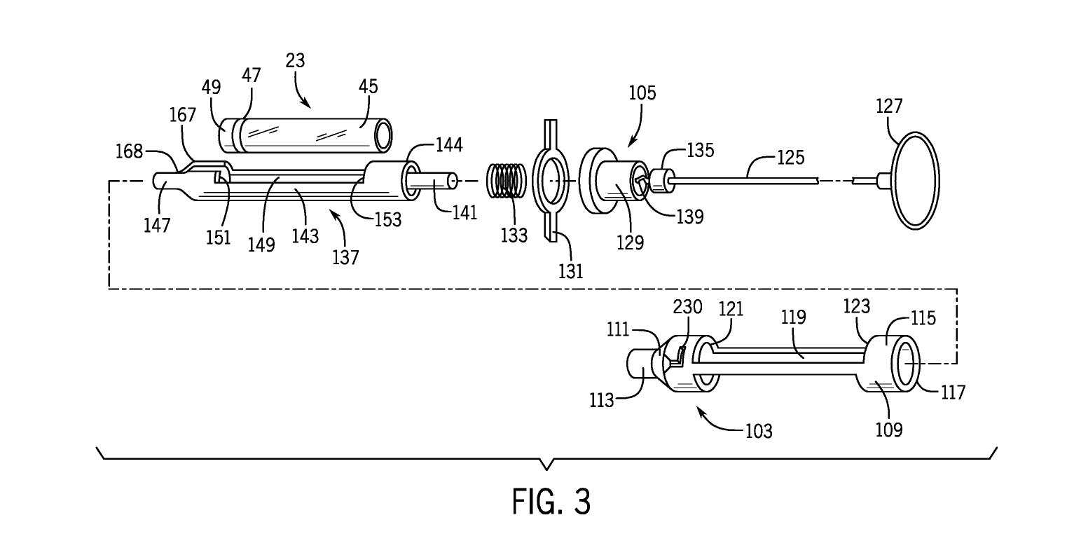

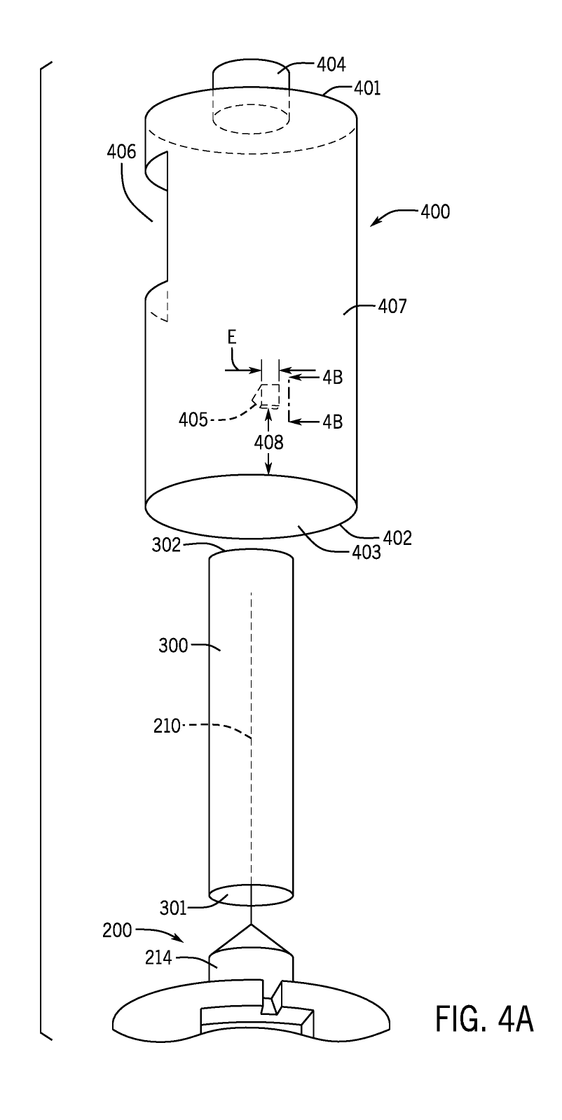

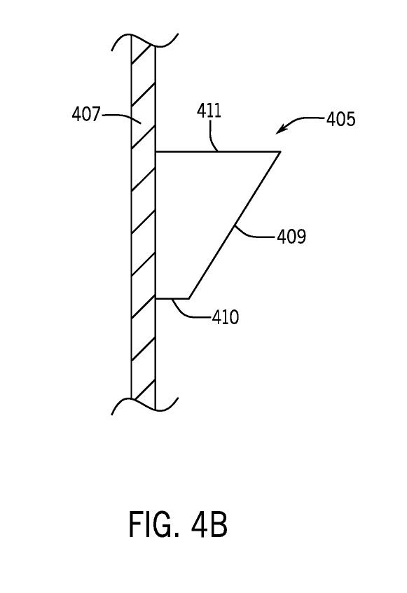

The improved syringe apparatus 101 of the present invention is shown in sectional view in FIG. 2 and in exploded view in FIG. 3. Syringe 101, which is configured to utilize the same conventional ampoule 23 as previously described, includes syringe body 103, plunger assembly 105, and a needle and cap assembly illustrated in FIGS. 4A, 4B, 5 and 6. The components of ampoule 23, having the same configuration as discussed with respect to the prior art syringe construction, will be referred to with the same, previously-used reference numbers.

Syringe body 103 includes cylindrical cage 109, having forward end 111 with narrowed syringe coupling 113, and rearward end 115 with thread 117. In order to save on material and lighten the weight, cylindrical cage 109 may be configured with mostly open-sided walls, as shown. Syringe body 103 also includes lateral aperture 119, which is bounded by front edge 121 and rear edge 123, and has a width sufficient to accommodate ampoule 23. Plunger assembly 105 includes plunger shaft 125, thumb ring 127, outer collar 129, finger grip collar 131, helical springs 133, pusher disk 135, and ampoule carrier frame 137. Hook 139 is affixed to pusher disk 135. Ampoule carrier frame 137 includes stein 141, central well 142, body 143, spring retainer 144 and needle ejector projection 147. Body 143 has an internal diameter which is greater than the outer diameter of ampoule 23, and has a lateral aperture 149 having front edge 151 and rear edge 153, which are spaced sufficiently apart to permit the ready insertion or release of ampoule 23, when ampoule carrier frame 137 is appropriately aligned in syringe body 103, so that ampoule 23 will be substantially surrounded by ampoule carrier frame 137, at least with respect to both rear end 45 and front end 47 of ampoule 23. Notch 167 (FIG. 3) extends forward from front edge 151 to forward notch edge 168. Body 143 also has an external diameter which is less than the internal diameter of syringe body 103, so as to enable ampoule carrier frame 137 to move forward and backward within syringe body 103.

Needle assembly, which, in the preferred embodiment shown in FIGS. 4A, 4B, 5 and 6, includes needle 210 that passes through needle retainer 200, and has substantially the same configuration as needle 89 previously described, with exterior end 211 and interior end 212, as further described below.

When syringe 101 is assembled, and held in an unloaded, resting position, for example, with exterior end 211 of needle 210 held upright, and with no pressure being exerted on plunger shaft 125, and no ampoule 23 loaded, ampoule carrier frame 137 is biased into an intermediate position, relative to syringe body 103, by spring 133, such that while rear end 45 of ampoule 23 may be inserted through aperture 119, and through aperture 149, to come into contact with rear wall 163 of ampoule carrier frame 137, front end 47 of ampoule 23 cannot clear the front edges 121, 151 of apertures 119, and 149, respectively. Upon pulling rearward on thumb ring 127, however, ampoule carrier frame 137 is drawn backward, against the bias of spring 133, and ampoule 23 is capable of clearing front edge 121 of aperture 119. In the above-described resting position of ampoule carrier frame 137, front edge 151 is not aligned with, but rather is forward of, front edge 121. Otherwise, if plunger shaft 125 is drawn backward, front edge 151 would move too far rearward relative to front edge 121, and ampoule 23 would be prevented from being fully inserted into body 143.

Upon release of plunger shaft 125, spring 133 pushes ampoule carrier frame 137 forward, causing seal 53 of ampoule 23 to contact, and be pierced by, interior end 212 of needle 210. Rear wall 163 of ampoule carrier frame 137 then moves forward to a position slightly rearward of the position it occupied when syringe 101 is in its unloaded configuration. The injection procedure is then executed.

Removal of ampoule 23 is accomplished by drawing backward on plunger shaft 125 with thumb ring 127, in the manner previously described. However, ampoule 23 is not provided with an opportunity to “hang up” on interior end 212 of needle 210, since, as plunger shaft 125 is drawn back, pusher disk 135 abuts rear wall 163 of ampoule carrier frame 137, and begins to drive ampoule carrier frame 137 backward, toward the ampoule insertion position. Since ampoule carder frame 137 completely surrounds ampoule 23, the inner front wall 165 of ampoule carrier frame 137 pushes against collar 49 of ampoule 23, and drives ampoule 23 off of interior end 212 of needle 210. Since the rearward movement of ampoule carrier frame 137 is controlled by pusher disk 135, whether hook 139 maintains a grip on plunger piston 55 has no effect on the removal procedure. Once ampoule 23 has been freed from interior needle end 212, and plunger shaft 125 has been fully withdrawn from within ampoule 23, ampoule 23 is free to slide within body 143 of ampoule carrier frame 137, and upon alignment of front edge 151 with front edge 121, the operator may simply turn syringe 101 over in his hand, can drop the spent ampoule into an appropriate waste receptacle.

An additional advantageous feature of the improved syringe 101 of the present invention is the provision of a mechanism for the selective automatic ejection of the used, disposable needle assembly. With concerns regarding the possible transmission of disease via syringe needles, it is especially desirable to provide a method of separating and ejecting a needle assembly from a syringe body without the operator having to directly handle the now-potentially contaminated used needle assembly and further prevent inadvertent contact with needle 210 after removal from the syringe body 103. Such a needle assembly is described further herein with respect to FIGS. 4, 5, and 8 below.

As previously mentioned, ampoule carrier frame 137 is provided with a notch 167, which, in the embodiment shown in FIGS. 2-3, extends from the forward edge 151 of lateral aperture 149, to forward notch edge 168. A corresponding axially extending projection 169 emanates from the inner front wall 171 of syringe body 103. When syringe apparatus 101 is assembled, notch 167 and projection 169 are aligned. When an ampoule 23 is installed in ampoule carrier frame 137, ampoule 23 is held in its forward most position within ampoule carrier frame 137, against the inside front surface 138. Collar 49 of ampoule 23 abuts rear edge 170 of projection 169, and ampoule carrier frame 137 is prevented from moving forward to its forward most possible position. Accordingly, needle ejector projection 147 is positioned rearwardly away from a needle retainer, such as needle retainer 200 described below with respect to FIGS. 4, 5, and 8. When pressure is applied to thumb ring 127, to force plunger piston 55 forward, the force is transmitted through plunger piston 55 and into the medicinal liquid 57. Part of this force is transferred to collar 49 and against the rear edge 170 of projection 169. At no time when an ampoule 23 is in ampoule carrier frame 137 can any thrusting force from plunger shaft 125 be transferred to ampoule carrier frame 137, and so ampoule carrier frame 137 is prevented from being moved forward.

When the operator is finished with the injection procedures, the ampoule may be removed, one-handed, using the process and apparatus described, and the needle assembly ejected in the following manner. When there is no ampoule 23 in ampoule carrier frame 137, the operator may simply press forward on thumb ring 127 until thumb ring collar 122 abuts a front surface 138 of ampoule carrier frame 137. The force is thus directly transferred to ampoule carrier frame 137, which is then free to move forward, with needle ejector projection 147 pushing a needle retainer off of syringe coupling 113. While the preferred embodiment shown employs one notch 167 and one projection 169, the alignment feature may effectively employ a greater number of corresponding notches and projections.

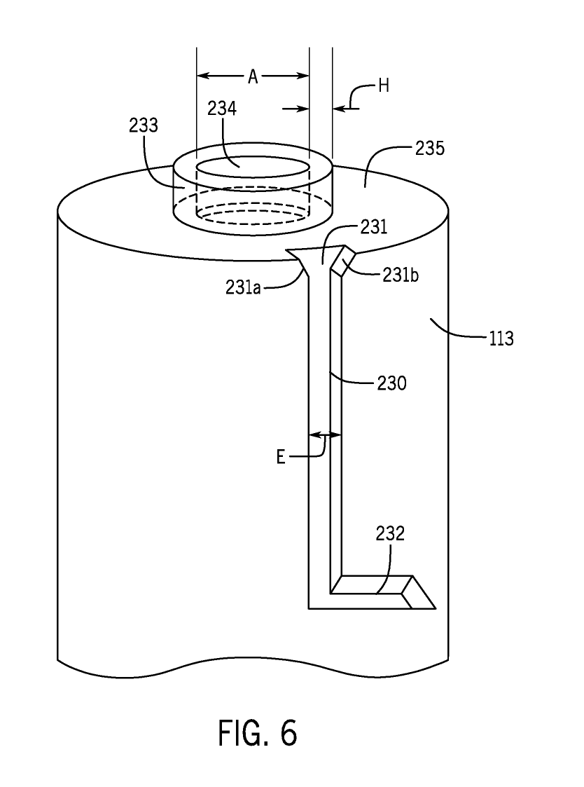

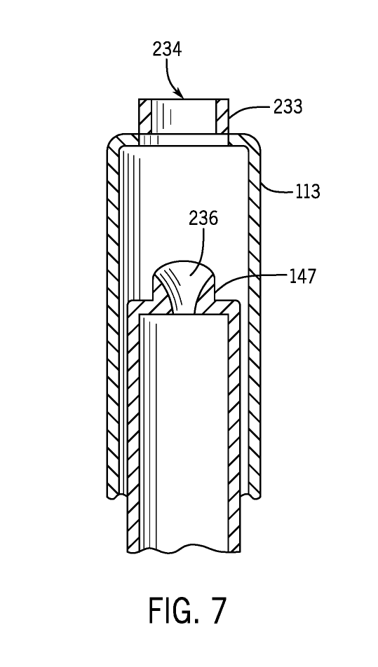

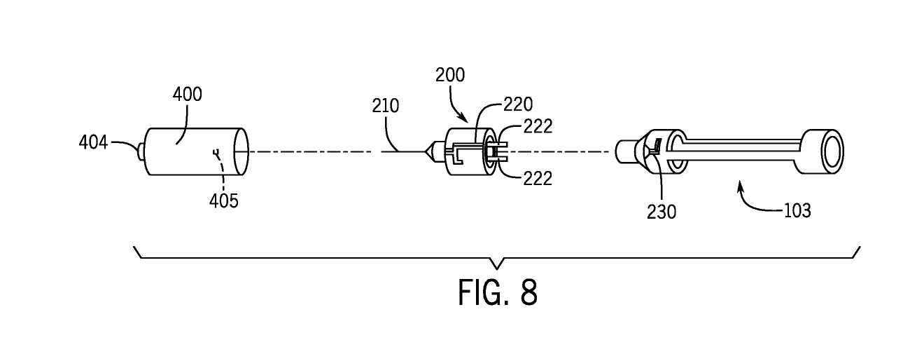

The syringe body 103 shown in FIG. 3 (also shown in FIG. 8 and in part in FIGS. 6 and 7) includes syringe coupling 113. Syringe coupling 113 includes a lower release channel 230, which is described in more detail below with respect to FIG. 6. The lower release channel 230 allows a guide pin (e.g., guide pin 405 of FIGS. 4A and 4B) of an outer needle cap (e.g., outer needle cap 400 of FIGS. 4A and 4B) to enter lower release channel 230 and lock onto syringe coupling 113. In this way, after use of ampoule 23 in the syringe, an outer cap can be placed around a needle assembly and locked onto syringe coupling 113. Ampoule 23 can then be disposed of and a new ampoule may be loaded into the syringe, all while the outer cap is locked into place around the needle assembly, preventing injury to the handler of the syringe from the needle and preventing contamination of the needle itself.

One particular feature of the present invention is the provision of an improved needle retainer and cap assembly for covering the syringe needle as shown in FIGS. 4A, 4B, 5 and 6. While it is desirable to cover the needle assembly with a cap, prior to mounting on the syringe body, to prevent injury, and to preserve the needle in a sterilized condition, it is also desirable to cover the needle tip after usage, in order to prevent injury and possible exposure to contamination or disease after use.

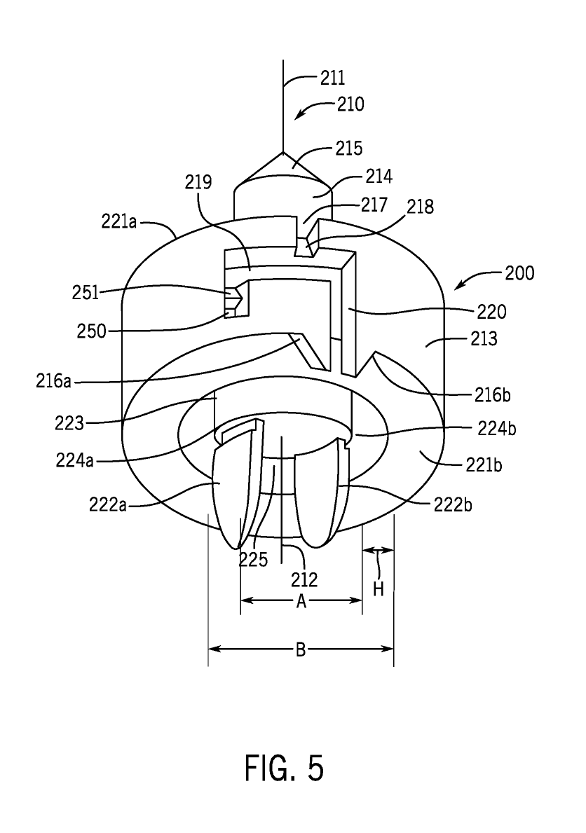

The improved cap apparatus of the present invention incorporates an inner cap 300 that encloses needle 210 prior to use and an outer needle cap 400 that covers needle 210 after use, as shown in FIGS. 4A, 4B, 5 and 6. In particular, FIG. 4A shows a perspective view of a needle assembly and cap assembly that may be used in combination with the syringe shown in FIGS. 2 and 3. FIG. 4B shows a side view of a guide pin on a side wall of an outer needle cap, such as the guide pin 405 of the outer needle cap 400 of FIG. 4A. FIG. 5 shows a perspective view of a needle assembly, such as the one shown in FIG. 4A. FIG. 6 shows a perspective view, partly in section, of a portion of a syringe body, such as syringe body 103 of FIG. 3.

FIG. 4A shows an inner cap 300 that is constructed as a substantially cylindrical member having an open end 301 and a closed end 302. Inner cap 300 is pre-positioned onto the forward. surface of needle retainer 200 and enclosing needle 210 with the open end 301 surrounding forward projecting member 214 (also shown in FIG. 5) and removably secured thereto by friction, and installed during manufacture and/or assembly of needle retainer 200. Outer needle cap 400 is constructed as a substantially cylindrical member having a top edge 401, bottom edge 402, an open end 403 and a side wall 407. The top, or forward end, of cap 400 may be open or partly closed with an opening sufficiently large to permit inner cap 300 to fit there through. A universal size outer cap is illustrated (FIG. 4A) having an open ended top collar 404 so as to accommodate and enclose inner caps of varying lengths. Guide pin 405 is positioned along an inward facing surface of side wall 407. Guide pin 405 is located a distance 408 from the bottom edge 402 of the outer needle cap 400. As further described below, the distance 408 is configured such that bottom edge 402 of the outer needle cap 400 extends past needle retainer 200 when the outer needle cap 400 is affixed to the needle retainer 200 and/or the syringe body 103. In this way, the outer needle cap 400 may extend past the interior end 212 of the needle 210, so as to further protect the interior end 212 from contamination or injuring a user when the needle retainer 200 is not affixed to the syringe body 103. As further described herein, guide pin 405 can slide into various channels of needle retainer 200 and/or syringe body 103 to lock and/or release outer needle cap 400 to needle retainer 200 and/or syringe body 103. Outer needle cap 400 includes an opening or “window” 406 located in a side wall thereof the purpose of which is described below.

Outer needle cap 400 may be pre-positioned onto needle retainer 200 and encloses inner cap 300 and needle 210 during manufacture and/or assembly of needle retainer 200 and moves from a covered to an open position as described herein. In this way, needle retainer 200, inner cap 300, needle 210, and outer needle cap 400 may be assembled as a complete package that can be used with a syringe such as the one shown in FIGS. 2 and 3. In this way, needle retainer 200, inner cap 300, needle 210, and outer needle cap 400 may be used disposably (e.g., with only one patient). After needle retainer 200, inner cap 300, needle 210, and outer needle cap 400 are used and disposed of as described herein, a new needle retainer, inner cap, needle, and outer cap package may be utilized with a syringe such as the one shown in FIGS. 2 and 3.

Guide pin 405 is further illustrated in FIG. 4B, which shows a partial cross-section of the outer needle cap 400 of FIG. 4A. In particular, FIG. 4B shows a portion of side wall 407 of the outer needle cap 400 in cross-section with guide pin 405 thereon. Guide pin 405 includes bottom surface 410 and top surface 411 which extend outward from side wall 407. Bottom surface 410 is located nearer to the bottom edge 402 of the outer needle cap 400, and top surface 411 is located nearer to top edge 401 of the outer needle cap 400. Top surface 411 extends out from side wall 407 further than bottom surface 410, forming an angled surface 409 of the guide pin 405. The shape of guide pin 405 allows the outer needle cap 400 to be locked to certain portions of the needle retainer 200, as discussed further below. In alternative embodiments, guide pin 405 may have variously shaped cross-sections, such as a substantially square cross-section, substantially rectangular, substantially triangular, or any other type of shape.

Needle retainer 200 illustrated in FIG. 5 comprises a generally cylindrical body 213 having a top surface 221a and bottom surface 221b. Vertical release channel 220 extends longitudinally along the side of body 213 extending from the bottom surface 221b to horizontal locking channel 219. Horizontal locking channel 219 extends along the side of body 213, perpendicular to vertical release channel 220. Horizontal locking channel 219 also extends between vertical release channel 220 and vertical locking channel 250. The vertical locking channel 250 includes retaining catch 251 on the inner (e.g., outer-facing) surface of vertical locking channel 250. An upper channel 217 extends between the top surface 221a. and horizontal locking channel 219. The width of vertical channel 220, horizontal locking channel 219 and vertical locking channel 250 generally correspond to the width of guide pin 405 of outer needle cap 400, as referenced by the letter “E”. The inner (e.g., outer-facing) surface of upper channel 217 includes retaining catch 218. The lower, or rearward, facing surface 221b of body 213 includes alignment pins 216a and 216b which cooperate with alignment groves 231a and 231b (shown in FIG. 6) of syringe coupling 113.

Needle retainer 200 has forward projecting members 214 and 215 from which upper end 211 of needle 210 projects, as well as rearward projecting inner post 223 which in the illustrated embodiment has a diameter smaller than the outer diameter of body 213 and substantially equal to that of member 215 so as to form cavity 225. In various embodiments, member 215 and rearward projecting inner post 223 may have different diameters.

Two prongs 222a and 222b project from post 223 and each has a substantially flat inner surface, and an arcuate outer surface. Needle retainer 200 is configured to fit to the forward end of syringe coupling 113 and is removably secured thereto by an interference fit between prongs 222a and 222b and the upper surface 235 of syringe coupling 113. In particular, arcuate outer surfaces of prongs 222a and 222b converge rearwardly, so that the prongs are pressed together by the inner edge of opening 234. Once prongs 222a and 222b have passed the opening 234, prongs 222a and 222b are free to spread slightly, and the inner edge of opening 234 prevents needle retainer 200 from being removed or falling out. FIG. 7 shows a sectional view of a portion of the syringe apparatus. Needle ejector projection 147 includes a forward-facing concave surface 236 shown in FIG. 7 which, when needle ejector projection 147 is brought into contact with prongs 222a and 222b, forces them toward each other, to enable grooves 224a and 224b to release the inner edge of opening 234, and permit ejection of needle retainer 200. In other words, needle ejector projection 147 can push prongs 222a and 222b together and then out of opening 234 to eject needle retainer 200 from syringe coupling 113. As shown in FIG. 5, upper channel 217 is not aligned vertically with vertical locking channel 220. Accordingly, while the outer needle cap 400 is positioned such that guide pin 405 is within horizontal locking channel 219 (whether placed there during manufacturing or by an end user), the user can push the needle retainer 200 onto the syringe coupling 113 by grasping the outer needle cap 400 and pushing the outer needle cap 400 and needle retainer 200 onto the syringe coupling together. This can occur because bottom surface 410 of guide pin 405, shown in FIG. 4B, can contact a side wall of horizontal locking channel 219 that is closest to bottom surface 221b of needle retainer 200. In this way, the needle retainer can be pushed onto the syringe coupling 113 without removing the outer needle cap 400 and exposing needle 210, further preventing injury and/or contamination.

Syringe coupling 113 (as shown in FIG. 6) includes a lower release channel 230 and lower locking channel 232 that extends along the side of syringe coupling 113 perpendicular to lower release channel 230. The upper or forward facing surface of syringe coupling 113 includes alignment groves 231a and 231b that cooperate with alignment pins 216a and 216b when needle retainer 200 is secured to syringe coupling 113 so as to align the vertical release channel 220 and lower release channel 230. The upper or forward facing surface of body 213 further includes upward or forward projecting collar 233 including opening 234. The forward projecting collar 233 in FIG. 6 represents the forward most portion of syringe body 103 of FIGS. 3 and 8. In alternative embodiments, syringe body 103 may include the lower release channel 230 on the forward most portion of syringe body 103 visible in FIGS. 3 and 8, and forward projecting collar 233 is not shown in FIGS. 3 and 8.

When assembled and in an unused state, needle retainer 200 is secured to syringe coupling 113 as described above, with vertical release channel 220 in alignment with lower release channel 230 and with collar 234 received into cavity 225 of needle retainer 200. As described above, needle retainer 200 may be secured to syringe coupling 113 by pushing the needle retainer 200 onto syringe coupling 113 via outer needle cap 400 because of the interference of guide pin 405 with the side wall of horizontal locking channel 219. Needle 210 is covered by inner cap 300, for example installed during manufacture of the needle retainer. In an initial unused state, outer needle cap 400 is positioned so as to enclose inner cap 300 and needle 210. Specifically, outer needle cap 400 overlies body 213 of needle retainer 200 such that guide pin resides in horizontal locking channel 219. To use the syringe, outer needle cap 400 is rotated to move guide pin 405 from horizontal locking channel 219 into vertical release channel 220. Outer needle cap 400 is then moved downward, or rearward, with guide pin 405 travelling along vertical release channel 220 and into lower release channel 230. Outer cap is then rotated further to move guide pin 405 from lower release channel 230 into lower locking channel 232 (shown in FIG. 6). The length of outer needle cap 400 and lower release channel 230 and orientation of lower locking channel 232 are designed such that when in an operative state, outer needle cap 400 no longer encloses inner cap 300. In such a state, at least a portion of inner cap 300 extends through open ended top collar 404 of outer needle cap 400. Inner cap 300 may then be manually removed and the syringe used as described above. Moreover, when outer needle cap 400 is “retracted” (e.g., when guide pin 405 is located in lower locking channel 232), the user is able to view ampoule 23 through opening 406 which would otherwise be covered and obscured by the side wall of outer needle cap 400. In this way, a user may advantageously determine whether ampoule 23 still has sufficient medicinal liquid 57 within for continued use. If not, the user can determine that the ampoule 23 should be removed and replaced by another ampoule. The dimensions of opening 406 can be tailored to the specific dimensions of needle 210, ampoule 23, lateral apertures 119 and 149 and/or ampoule carrier frame 137. When the injection is complete, outer needle cap 400 may be returned to its initial state by rotating the cap to move guide pin 405 from lower locking channel 232, into lower release channel 230 toward being moved into upper horizontal locking channel 219. Retaining catch 218 prevents guide pin 405 from exiting horizontal locking channel 219 and being removed from needle retainer 200. Accordingly, outer needle cap 400 now encloses needle 210 thereby preventing unwanted contact therewith.

A distance between the guide pin 405 and the top edge 401 of the outer needle cap 400 is configured such that when the guide pin 405 is in the lower locking channel 232, the needle 210 is sufficiently exposed such that it may be easily used by an operator. In other words, the dimensions of the outer needle cap 400 are configured such that the needle may be used without hindering the functioning of the needle and without blocking the operator's view. The distance between the guide pin 405 may also be configured to be a proper distance with respect to the configuration of the lower release channel 230 and the lower locking channel 232 of the syringe body 103. In other words, because the guide pin 405 moves through the lower release channel 230 and the lower locking channel 232 to expose the needle 210 for use, the length and placement of the lower release channel 230 and the lower locking channel 232 as well as the position of the guide pin 405 and the length between the guide pin 405 and the top edge of the outer needle cap 400 all are factors in providing for the outer needle cap 400 to both cover the needle 210 properly when required and expose the needle 210 when required. Accordingly, various dimensions and lengths of the lower release channel 230 and the lower locking channel 232, the placement of the guide pin 405 on the outer needle cap 400, and dimensions of the outer needle cap 400 all cooperate for a properly functioning outer needle cap 400. Accordingly, each of those aspects may be configured in various ways to provide the advantageous aspects described herein: an outer needle cap that covers and protects a needle when desired and exposes a needle for use when desired. In addition, the syringe body 103 as shown, for example, in FIG. 3 is not shown to scale, and may be configured in various ways to accommodate differently sized release and/or locking channels. For example, the front edge 121 of the syringe body 103 may extend further into the lateral aperture 119 to allow for a longer lower release channel 230. In another example, the lateral aperture 119 may not extend through both sides of the syringe body 103 to accommodate different sized lower release channel 230 and/or lower locking channel 232. Accordingly, it is contemplated herein that the syringe body 103, the lower release channel 230, the lower locking channel 232, the outer needle cap 400, and/or the guide pin 400 may all he configured in various ways to provide the advantages described herein.

Locking channels 219 and 232 extend in opposite directions from one another with respect to vertical release channel 220 and lower release channel 230. In various embodiments of the invention, locking channels 219 and 232 may extend in directions opposite to that depicted in the Figures, or alternatively, in the same direction as each other. While the present disclosure contemplates outer needle cap 400 being preinstalled on the needle retainer, it could be installed at the time of use by sliding guide pin 405 through the upper portion 217 of vertical release channel 220 and retaining catch 218. In an embodiment, outer needle cap 400 may be formed out of transparent or partially transparent material, such as plastic, so that a user may more easily see where guide pin 405 is located within channels of syringe coupling 113 and/or needle retainer 200.

FIG. 8 is an exploded perspective view of syringe body 103, needle assembly including needle 210 and needle retainer 200, and outer needle cap 400. Inner cap 300 is not shown in FIG. 8 for simplicity, though in various embodiments inner cap 300 may exist between needle retainer 200 and outer needle cap 400. In other embodiments, inner cap 300 may already have been removed. FIG. 8 demonstrates, in particular, how syringe body 103, the needle assembly, and outer needle cap 400 align and function together. Also not shown in FIG. 8 for simplicity is opening 406 of outer needle cap 400 and alignment pins 216a and 216b of needle retainer 200.

Needle retainer 200 includes prongs 222 that allow needle retainer 200 to lock to syringe body 103. When locked to syringe body 103 as described herein, vertical release channel 220 and lower release channel 230 align. In this way, guide pin 405 of needle cap 400 can slide between vertical release channel 220 and lower release channel 230. When guide pin 405 of needle cap 400 is in horizontal locking channel 219 of needle retainer 200, needle cap 400 covers needle 210, protecting a user from a sharp end of needle 210 and protecting needle 210 from contamination. Guide pin 405 may then be moved as described herein from horizontal locking channel 219 to vertical release channel 220, and subsequently to lower release channel 230. From lower release channel 230, guide pin 405 may then be moved into lower locking channel 232, locking needle cap 400 onto syringe body 103.

When needle cap 400 is moved from horizontal locking channel 219 to lower locking channel 232, needle 210 extends through open ended top collar 404 of needle cap 400. In this way, needle 210 is exposed and may be used for injections as described herein. Once an injection is complete or a syringe needs to be reloaded with a different ampoule, needle cap 400 may be moved such that guide pin 405 is moved from being locked into syringe body 103 to being locked into needle retainer 200, allowing for needle retainer 200 to be disposed of or an ampoule to be reloaded.

In an alternative embodiment, open ended top collar 404 may be shaped such that it has an interference fit with forward projecting member 214 of needle retainer 200. In this way, when needle cap 400 is retracted so that the syringe and needle 210 is used, open ended top collar 404 fits snugly around forward projecting member 214 to lock needle cap into its retracted position. in such embodiments, lower locking channel 232 and/or lower release channel 230 may be omitted. That is, guide pin 405 may not need to be locked or otherwise guided when needle cap 400 is locked because of an interference fit between open ended top collar 404 and forward projecting member 214. In such embodiments, inner cap 300 may be shaped such that it has a widest diameter that is smaller than a diameter of an opening of open ended top collar 404. For example, such an inner cap may interference fit onto a portion of forward projecting member 214 that is narrower than the opening of open ended top collar 404. Accordingly, forward projecting member 214 may be constructed to have more than one diameter such that both a relatively smaller diameter inner cap 300 and a relatively larger diameter open ended top collar 404 may fit securely over forward projecting member 214, but still allowing for inner cap 300 to be removed through open ended top collar 404 as described herein.

The guide pin 405 may also be moved through horizontal locking channel 219 into vertical locking channel 250. Vertical locking channel 250 includes retaining catch 251 on the inner (e.g., outer-facing) surface of vertical locking channel 250. This retaining catch 251 presents interference for guide pin 405. In particular an angled surface 409 of guide pin 405 interacts with an angled surface of retaining catch 251 to cause the interference. The interference may cause outer cap 400 and/or needle retainer 200 to deform slightly such that guide pin 405 may move past retaining catch 251. Once guide pin 405 has moved past retaining catch 251, the guide pin 405 cannot move past retaining catch 251 again, thereby locking the outer needle cap 400 in place. Locking outer needle cap 400 in place in vertical locking channel 250 may be done by a user after finishing using a particular needle 210 and needle retainer 200. Once the outer needle cap 400 is locked, the entire assembly of the needle 210, needle retainer 200 and outer needle cap 400 may be removed from syringe coupling 113 and disposed of. In contrast, if a user merely wants to reload ampoules and continue using a needle 210 and needle retainer 200, the user can move guide pin 405 into horizontal locking channel 219 while ampoules are exchanged. In this way, guide pin 405 is not permanently locked in place and can again be moved into lower release channel 230 of syringe coupling 113 so that needle 210 may be used for further injections.

In alternative embodiments, the channels of needle retainer 200 and syringe body 103 may have varying depths. For example, at an end of one or both of horizontal locking channel 219 and lower locking channel 232, the channels may have a depth slightly shallower than a depth of guide pin 405. In such examples, this shallower depth may provide a tighter fit for outer needle cap 400 when guide pin 405 is slid into the end of either of horizontal locking channel 219 or lower locking channel 232. This may help lock outer needle cap 400 more effectively to better secure outer needle cap 400 to protect needle 210 or make sure outer needle cap 400 does not move and interfere with an injection. In various embodiments, this depth may change at an incline to provide a smooth transition between a locking position of outer needle cap 400 and portions of channels with deeper depths where outer needle cap 400 can move freely without interference. Such a configuration of horizontal locking channel 219 may also be beneficial where, in an embodiment, needle retainer 200 does not have vertical locking channel 250 with retaining notch 251.

In various embodiments, widths of channels may also be varied to provide interference for guide pin 405 and outer needle cap 400 to better lock outer needle cap 400 into place. For example, at an end of one or both of horizontal locking channel 219 and lower locking channel 232, the channels may have a width slightly narrower than a dimension “E” of guide pin 405. In such examples, this narrower width may provide a tighter fit for outer needle cap 400 when guide pin 405 is slid into the end of either of horizontal locking channel 219 or lower locking channel 232. Such a configuration of horizontal locking channel 219 may also be beneficial where, in an embodiment, needle retainer 200 does not have vertical locking channel 250 with retaining notch 251.

In various embodiments, varying widths and or depths of channels in needle retainer 200 and/or syringe body 103 may be utilized in such a way that horizontal locking channel 219 and/or lower locking channel 232 may be omitted. For example, a width and/or depth of vertical release channel 220 may get smaller at a forward end of needle retainer 200 (e.g., at an end closer to forward projecting member 214) to lock outer needle cap 400 into place without the use of horizontal locking channel 219. In a similar example, a width and/or depth of lower release channel 230 may get smaller at rearward end 115 of syringe body 103 to lock outer needle cap 400 into place without the use of lower locking channel 232.

In various embodiments, an outer needle cap 400, syringe body 103, and needle retainer 200 may be configured in other ways that permit outer needle cap 400 to slide over portions of syringe body 103 and needle retainer 200 to cover or uncover needle 210 as described herein. For example, instead of outer needle cap 400 having a guide pin 405 that extends into the various channels of syringe body 103 and needle retainer 200, an inside surface of outer needle cap 400 may include guide channels through which one or more guide tracks and/or pins of syringe body 103 and needle retainer 200 can slidably move. That is, the guide pin of outer needle cap 400 and the channels of syringe body 103 and needle retainer 200 may be reversed but in a way that still provides similar functionality of outer needle cap 400 as described herein.

Accordingly, an example improved cap apparatus for use and in combination with a syringe apparatus, each such assembly including a needle retainer and a needle member. The improved cap apparatus includes a substantially elongated inner cap member having a front end, a rear end and a longitudinal axis. The inner cap member is operably configured to substantially surround a needle member and at least a portion of needle retainer. The improved cap apparatus includes a substantially elongated outer cap member having a front end, a rear end and a longitudinal axis. The outer cap member is operably configured to substantially surround the inner cap member and at least a portion of needle retainer. The outer cap member includes a guide pin slideably postionable within a release channel and locking channel of the needle retainer. An associated syringe apparatus includes a lower locking channel and a lower release channel aligned with the release channel of the needle retainer. The outer cap may be moved from a position covering the inner cap to a position exposing the inner to cap to permit its removal prior to using the syringe apparatus. The outer cap may also be moved to return the outer cap to its initial position and locked in place after using the syringe apparatus to prevent contact with the needle.

The foregoing description and drawings merely explain and illustrate the invention, and the invention is not limited thereto except insofar and the appended claims are so limited, as those skilled in the art who have the disclosure before them will be able to make modifications and variations therein without departing from the scope of the invention.

What is claimed is:

1. A syringe cap and needle apparatus comprising:
a needle retainer;
a needle member secured within the needle retainer, the needle member further comprising an exterior needle end that extends outside of a forward end of the needle retainer; and
an inner cap comprising a substantially elongated outer body with a front end, a rear end and a longitudinal axis, wherein the inner cap is configured to substantially surround the exterior needle end and at least a portion of needle retainer;
an outer cap comprising a substantially elongated outer body with a front end, a rear end and a longitudinal axis, wherein:
the outer cap comprises a guide pin slideably postionable within at least one channel of the needle retainer, such that the outer cap is configured to slideably move with respect to the needle retainer between at least a first position and a second position;
in the first position, the outer cap substantially surrounds the exterior needle end and the inner cap;
in the first position and the second position, the outer cap substantially surrounds at least a portion of the needle retainer; and
in the second position, at least a portion of the exterior needle end and at least a portion of the inner cap are not surrounded by the outer cap.

2. The syringe cap and needle apparatus of claim 1, wherein the at least one channel of the needle retainer comprises a release channel and a locking channel, and further wherein the guide pin is configured to lock the outer cap into the locking channel in the first position.

3. The syringe cap and needle apparatus of claim 2, wherein:
the needle retainer is further configured to attach to a syringe apparatus comprising a lower release channel and a lower locking channel;
the lower release channel is configured to align with the release channel of the needle retainer such that the guide pin can move between the release channel of the needle retainer and the lower release channel of the syringe apparatus; and
the guide pin is further configured to lock the outer cap into the lower locking channel of the syringe apparatus in the second position.

4. The syringe cap and needle apparatus of claim 3, wherein the guide pin is further configured to move from the locking channel of the needle retainer at the first position to the release channel of the needle retainer, then to the lower release channel of the syringe apparatus, then to the lower locking channel of the syringe apparatus at the second position.

5. The syringe cap and needle apparatus of claim 3, wherein while the outer cap is in the second position, at least part of the inner cap is exposed such that the inner cap is removable to permit its removal from the syringe cap and needle assembly.

6. A syringe cap and needle apparatus comprising:
a needle retainer;
a needle member secured within the needle retainer, the needle member further comprising an exterior needle end that extends outside of a forward end of the needle retainer; and
a cap, wherein:
the cap is configured to slideably move with respect to the needle retainer between at least a first position and a second position;
in the first position, the cap substantially surrounds the exterior needle end;
in the first position and the second position, the cap substantially surrounds at least a portion of the needle retainer; and
in the second position, at least a portion of the exterior needle end is not surrounded by the cap.

7. The syringe cap and needle apparatus of claim 6, wherein the cap comprises an substantially circular opening at a rear end that has a diameter that is wider than a width of the needle retainer.

8. The syringe cap and needle apparatus of claim 6, wherein in the second position the cap substantially surrounds the needle retainer.

9. The syringe cap and needle apparatus of claim 6, wherein the cap is substantially elongated, having a front end, a rear end, and a longitudinal axis.

10. The syringe cap and needle apparatus of claim 10, wherein the cap is an outer cap, and the apparatus further comprises an inner cap that is substantially elongated, having a front end, a rear end and a longitudinal axis, wherein:
the inner cap is configured to substantially surround the exterior needle end and at least a portion of the needle retainer and
in the first position, the outer cap is configured to substantially surround at least a portion of the inner cap.

11. The syringe cap and needle apparatus of claim 11, wherein in the first position, the outer cap is configured to substantially surround the inner cap.

12. The syringe cap and needle apparatus of claim 11, wherein the outer cap comprises an opening in the front end configured such that, in the first position or the second position, a portion of the inner cap extends through the opening.

13. The syringe cap and needle apparatus of claim 11, wherein the inner cap is removable from the needle retainer while the outer cap is in the second position.

14. The syringe cap and needle apparatus of claim 6, wherein the cap further comprises a guide pin slideably postionable within a release channel of the needle retainer.

15. The syringe cap and needle apparatus of claim 13, wherein the release channel is configured to align with a lower release channel of a syringe apparatus, such that the guide pin is slideably moveable between the release channel and the lower release channel.

16. A method comprising:
moving an outer needle cap from a first position to a second position, wherein:
the outer cap is configured to slideably move with respect to a needle retainer between the first position and the second position,
in the first position, the outer cap substantially surrounds an exterior needle end of a needle member secured within the needle retainer and the outer cap substantially surrounds an inner needle cap, and
in the first position and the second position, the outer cap substantially surrounds at least a portion of the needle retainer;
removing, after the outer cap is moved into the second position, the inner needle cap from a position affixed to the needle retainer, thereby exposing the exterior needle end;
engaging a syringe apparatus operatively connected to the needle retainer to perform an injection; and
after the injection, moving the outer cap from the second position back to the first position.

17. The method of claim 16, further comprising, before moving the outer cap from the first position to the second position, affixing a needle retainer assembly to the syringe apparatus, wherein the needle retainer assembly comprises the needle member, the needle retainer, the outer cap, and the inner cap.

18. The method of claim 16, further comprising, after moving the outer cap from the second position back to the first position, removing a first ampoule from the syringe apparatus and replacing the first ampoule with a second ampoule.

19. The method of claim 18, further comprising moving, after replacing the first ampoule with the second ampoule, the outer cap from the first position to the second position to perform a second injection.

20. The method of claim 16, further comprising, after moving the outer cap from the second position back to the first position, detaching the needle retainer and the outer cap from the syringe apparatus.
